(19) United States
(12) Patent Application Publication
(10) Pub. No.: US 2026/0130932 A1
(43) Pub. Date: May 14, 2026

(54) NOVEL COMBINATION OF RIFAXIMIN AND CLARITHROMYCIN FOR TREATING MULTIDRUG-RESISTANT MYCOBACTERIUM ABSCESSUS

(71) Applicant: Massachusetts Institute of Technology, Cambridge, MA (US)

(72) Inventors: Peter C. DEDON, Singapore (SG); Boon Chong GOH, Singapore (SG)

(21) Appl. No.: 19/115,743

(22) PCT Filed: Sep. 26, 2023
(86) PCT No.: PCT/US2023/075161

§ 371 Date: Mar. 26, 2025

Related U.S. Application Data
(60) Provisional application No. 63/409,928, filed on Sep. 26, 2022.

(51) Int. Cl. A61K 31/7048; A61K 31/437; A61P 31/04

(52) U.S. Cl. CPC A61K 31/7048; A61K 31/437; A61P 31/04

(57) ABSTRACT

The present invention relates to a novel synergistic combination of rifaximin and clarithromycin. The invention also relates to a kit comprising such combination, and such combination for use as pharmaceuticals, for instance in the treatment of bacterial diseases, including diseases caused by pathogenic mycobacteria such as clarithromycin-resistant and clarithromycin non-resistant non-tuberculosis mycobacteria.

### CROSS-REFERENCE TO RELATED APPLICATIONS

The present application is a filing under 35 U.S.C. 371 as the National Stage of International Application No. PCT/US2023/075161, filed Sep. 26, 2023, entitled “NOVEL COMBINATION OF RIFAXIMIN AND CLARITHROMYCIN FOR TREATING MULTIDRUG-RESISTANT MYCOBACTERIUM ABSCESSUS,” which claims priority to U.S. Provisional Application No. 63/409,928 filed with the United States Patent and Trademark Office on Sep. 26, 2022 and entitled “NOVEL COMBINATION OF RIFAXIMIN AND CLARITHROMYCIN FOR TREATING MULTIDRUG-RESISTANT MYCOBACTERIUM ABSCESSUS,” both of which are incorporated herein by reference in their entirety for all purposes.

### FIELD OF THE INVENTION

The present invention relates to a novel synergistic combination of rifaximin and clarithromycin. The invention also relates to a kit comprising such combination, and such combination for use as pharmaceuticals, for instance in the treatment of bacterial diseases, including diseases caused by pathogenic mycobacteria such as clarithromycin-resistant and clarithromycin non-resistant non-tuberculosis mycobacteria.

### BACKGROUND OF THE INVENTION

Infections caused by nontuberculous mycobacteria (NTM) are being recognized as a fast-rising health threat globally, especially in the context of intractable pulmonary infections. Among NTMs, the Mycobacterium avium and Mycobacterium abscessus complexes are the most commonly implicated in human infections. M. abscessus is known to cause pulmonary infections in patients with immune deficiencies or with underlying lung conditions, such as cystic fibrosis, bronchiectasis, or chronic obstructive pulmonary disease (COPD). Further, demonstrated transmission of M. abscessus between cystic fibrosis patients has increased the urgency to identify novel treatments for this pathogen. In addition to lung diseases, Mycobacterium abscessus has also been implicated in severe infections in cutaneous, joint, soft tissue, and surgical sites. However, chemotherapeutic options for treating M. abscessus infections are limited because the bacterium is intrinsically resistant to most antibiotics. M. abscessus infections are treated by a multi-drug regimen consisting of clarithromycin (Clr), amikacin, and either cefoxitin, imipenem, or tigecycline. However, such treatments are complicated by the ability of some M. abscessus sub-species to acquire phenotypic resistance to Clr upon repeated exposure to the drug. The presence of erm(41) gene is responsible for inducible resistance to macrolides. Since Clr is currently the only highly effective oral antibiotic, resistance to Clr is of great concern.

However, there is still a need to explore new regimes for the treatment of mycobacterium related infections, especially to overcome the problem of Clr resistance in such treatment.

### SUMMARY OF THE INVENTION

According to the present invention, rifaximin (Rfx) can be used as a Clr potentiator that overcomes Clr resistance in NTM. The combination of Rfx+Clr is bactericidal in vitro and efficacious in a zebrafish embryo infection model. While Rfx is a non-systemic antibiotic that was approved for treating gastrointestinal bacterial infections, the inventors propose to repurpose Rfx in combination with Clr to treat M. abscessus lung, gastrointestinal and/or topical infections. The inventors found that the combination of Rfx and Clr is synergistic in activity against clarithromycin-resistant and clarithromycin non-resistant mycobacteria.

Thus, in a fist aspect of the invention, there is provided an antibiotic combination, comprising a therapeutically effective amount of clarithromycin and rifaximin, wherein the combination is bactericidal to clarithromycin-resistant and clarithromycin non-resistant mycobacteria.

In some embodiments, the mycobacterium is Mycobacterium abscessus or Mycobacterium avium. Preferably, the mycobacterium is Mycobacterium abscessus. More preferably, the mycobacterium is clarithromycin-resistant Mycobacterium abscessus. In some embodiments, the antibiotic combination has a fractional inhibitory concentration index (FICI)≤0.5 for Mycobacterium abscessus.

In some embodiments, the antibiotic combination is formulated for administration of:
i) clarithromycin at a dosage in the range of 0.5 μg/ml to 75 μg/ml, and rifaximin at a dosage in the range of 1 μg/ml to 64 μg/ml, or
ii) rifaximin and clarithromycin each independently at a dosage in the range of 0.5 mg/kg to 50 mg/kg, preferably 5 mg/kg to 20 mg/kg, more preferable at about 10 mg/kg.

In some embodiments, the clarithromycin and the rifaximin may be formulated as a single composition or as separate compositions. Preferably, the composition further comprises a pharmaceutically acceptable carrier and/or excipient. In some embodiments wherein the clarithromycin and the rifaximin are formulated as a single composition, said composition is for nasal, oral or topical administration. In some embodiments wherein the clarithromycin and the rifaximin combination are formulated as separate compositions, said separate compositions may be administered in conjunction simultaneously or sequentially, and each composition may be administered by nasal, oral or topical routes, and the administration routes of said separate compositions may be the same or different.

In some embodiments, the antibiotic combination is for use in the treatment of non-tuberculous mycobacteria (NTM) infections.

In some embodiments, the antibiotic combination comprises Rfx and Clr in amounts that act synergistically to treat clarithromycin-resistant and clarithromycin non-resistant mycobacteria infections.

In a second aspect of the invention, there is provided the antibiotic combination of the first aspect of the present invention for use in the treatment of clarithromycin-resistant and clarithromycin non-resistant mycobacteria, including non-tuberculous mycobacteria (NTM) infection. Preferably, the clarithromycin and rifaximin combination is together in a single composition.

In a third aspect of the invention, there is provided the use of the antibiotic combination of the first aspect in the manufacture of a medicament for the treatment of non-tuberculous mycobacteria infection.

In some embodiments, the infection is a pulmonary infection, a gastrointestinal infection or a skin infection. In some embodiments, the infection is caused by Mycobacterium abscessus and/or Mycobacterium avium. In such embodiments the Mycobacterium abscessus and/or Mycobacterium avium is clarithromycin-resistant or non-resistant, preferably clarithromycin-resistant. In some embodiments, the infection is caused by clarithromycin-resistant Mycobacterium abscessus.

In some embodiments, the medicament comprises a synergistic combination of clarithromycin and rifaximin.

In a fourth aspect of the invention, there is provided a method of treating Mycobacterium infection in a subject, comprising administering a therapeutically effective combination of clarithromycin and rifaximin according to the first aspect to said subject. In some embodiments, the clarithromycin and the rifaximin are administered as a single composition. In some embodiments, the clarithromycin and the rifaximin are administered as separate compositions. The administration may be simultaneous or sequential.

In some embodiments, the infection is a pulmonary infection, a gastrointestinal infection or a skin infection.

In some embodiments, the infection is caused by Mycobacterium abscessus and/or Mycobacterium avium. In some embodiments, the infection is caused by clarithromycin-resistant Mycobacterium abscessus. In some embodiments, administration of the clarithromycin and the rifaximin results in synergistic inhibition of said Mycobacterium infection.

In a fifth aspect of the invention, there is provided a kit for treating Mycobacterium infection comprising the antibiotic combination of clarithromycin and rifaximin of the first aspect. In some embodiments, the kit may comprise a composition comprising an amount of clarithromycin in combination with an amount of rifaximin that, together, are therapeutically effective.

In some embodiments, the infection is a pulmonary infection, a gastrointestinal infection or a skin infection. In some embodiments, the infection is a non-tuberculous mycobacteria (NTM) infection. In some embodiments, the infection is caused by;
i) Mycobacteriumm abscessus and/or Mycobacterium avium; or
ii) clarithromycin-resistant Mycobacterium abscessus.

In some embodiments, the kit comprises the following components: (i) a composition including clarithromycin in admixture with a pharmaceutically acceptable carrier; and (ii) a composition including rifaximin in admixture with a pharmaceutically acceptable carrier, wherein the components (i) and (ii) are each provided in a form that is suitable for administration in conjunction with the other, such as for simultaneous or sequential administration. Preferably, the combination of Rfx and Clr is synergistic.

### BRIEF DESCRIPTION OF THE DRAWINGS

Certain embodiments of the present disclosure are described more fully hereinafter with reference to the accompanying drawings.

FIG. 1 shows the MIC of M. abscessus strain M422 before and after 3 days of induction with 0.25-4 μg/ml of clarithromycin.

FIG. 2 shows the scatter plots of the screening of 2,252 compounds. The compounds that resulted in >90% growth inhibition (below the black line) of M. abscessus are defined as hits. The combination of clarithromycin (Clr) and vancomycin (Van) was used as a positive control.

FIG. 3 shows the dose response of rifaximin on M. abscessus strain M422 with and without the presence of 4 μg/ml of clarithromycin.

FIG. 4 shows the isobolograms for Rfx and Clr against M. abscessus and M. avium strains. The dotted line indicates FICI≤0.5. The FIC data points that fall below the dotted line are defined as synergistic. All three strains of M. abscessus were pre-induced whereas the M. avium strain was uninduced.

FIG. 5 shows the MBC₉₀ and MIC determination of the combination of Clr and Rfx on M. abscessus ATCC 19977. The solid grey line indicates MBC₉₀, and the dashed black line indicates the limit of detection. Statistical significance was determined by a two-tailed Student's t-test with Welch's correction. * p<0.05; ** p<0.01. The experiments were carried out in duplicates; error bars represent the standard deviation.

FIGS. 6A-6B shows the synergistic effects of rifaximin with clarithromycin to reduce bacterial load of M. abscessus in infected zebrafish larvae. Each dot plotted represents larvae homogenized together. (FIG. 6A) Infected fishes treated with various concentrations of Clr. (FIG. 6B) The bacterial burdens of animals treated with rifaximin only were compared with those of animals treated with rifaximin and clarithromycin in combination. 3 μg/ml Bedaquiline was used as a control as it has demonstrated efficacy against M. abscessus in zebrafish embryos (2). Note that each dot plotted represents 5 larvae homogenized together. Fish were infected in the hindbrain ventricle at 48 hpf, and treatment began at 72 hpf. Treatment was maintained for four days with daily water changes before the fish were assessed for bacterial burden.

### DETAILED DESCRIPTION OF THE INVENTION

Further details of the invention will now be described with reference to the following non-limiting examples. Unless otherwise defined herein, technical and scientific terms used in the present description have the meanings that are commonly understood by those of ordinary skill in the art.

### A. Definitions

As used herein, the singular forms “a,” “an,” and “the” include plural referents unless the context clearly dictates otherwise.

As used herein, the term “comprising” may include the embodiments “consisting of” and “consisting essentially of”. The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

As used herein, the term “combination” refers to two agents (such as Clr and Rfx), and means that said two agents are administered in conjunction. Thus, the agents may be presented (i.e. formulated) either as a combined preparation (i.e. presented as a single composition including both agents) or, alternatively, may be presented as separate compositions, wherein at least one of those compositions comprises one agent (such as Clr) and at least one comprises the other agent (such as Rfx). When the two agents are in separate compositions they are administered, if not simultaneously, then sequentially within a timeframe, so that they both are available to act therapeutically within the same time frame.

As used herein, the term “simultaneously” is used to mean that the two agents are administered concurrently. The term “sequentially” means that one agent is administered within 5 minutes, 10 minutes or a matter of hours after the other agent provided the circulatory half-life of the first administered agent is such that they are both concurrently present in therapeutically effective amounts. The time delay between administrations of the two agents will vary depending on the exact nature of the agents, the interaction there between, and their respective half-lives.

The phrase “pharmaceutically acceptable”, as used in connection with compositions described herein, refers to molecular entities and other ingredients of such compositions that are physiologically tolerable and do not typically produce untoward reactions when administered to a subject (e.g., a human).

As used herein, the term “therapeutically effective amount” or “efficacious amount” of a drug refers to an amount of the drug that is an amount sufficient to obtain a pharmacological response such as inhibiting a biological target (e.g., inhibiting the growth or spread of a bacteria such as mycobacteria); or alternatively, is an amount of the drug that, when administered to a subject with a specified disorder or disease, is sufficient to have the intended effect, e.g., treatment, alleviation, amelioration, palliation or elimination of one or more manifestations of the specified disorder or disease in the subject. A therapeutic effect does not necessarily occur by administration of one dose, and may occur only after administration of a series of doses. Thus, a therapeutically effective amount may be administered in one or more administrations. The therapeutically effective amount will depend in part on the nature of the drug, the manner and route of administration, the stage and severity of the disease being treated, the weight and general state of health of the subject, and the judgment of the prescribing physician. Further, when the term “therapeutically effective amount” or “efficacious amount” is used to refer to a synergistic combination of antibiotic compounds, it means that each of said compounds is provided at an amount such that the combination, as a whole, would provide therapeutic effects. It would be understood that, for said synergistic combination of antibiotic compounds, the amount of one or both of said compounds in the combination, if used alone at the dose in the combination, may not provide a therapeutic effect.

As used herein, the term “subject” refers to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, or infant). A mammal can be male or female. In some embodiments, a subject is a human. In some embodiments, a subject has or is diagnosed of having a particular disease, for example, non-tuberculous mycobacteria infection.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

### B. Combination of Rifaximin and Clarithromycin

The invention provides a antibiotic combination comprising an amount of clarithromycin and an amount of rifaximin, wherein the combination is bactericidal to clarithromycin-resistant and clarithromycin non-resistant mycobacteria. Said combination may be formulated as a single composition or into separate compositions, which can be administered simultaneously or sequentially in therapeutically effective amounts.

In some embodiments, the mycobacterium is Mycobacterium abscessus or Mycobacterium avium. Preferably, the mycobacterium is Mycobacterium abscessus. More preferably, the mycobacterium is clarithromycin-resistant Mycobacterium abscessus. In some embodiments, the composition has a fractional inhibitory concentration index (FICI)≤0.5 for Mycobacterium abscessus.

In some embodiments, the amount of clarithromycin is in the range of 0.5 μg/ml to 75 μg/ml. For examples, the amount of clarithromycin may be in the range of 0.5 μg/ml to 65 μg/ml, 0.5 μg/ml to 55 μg/ml, 0.5 μg/ml to 45 μg/ml, 0.5 μg/ml to 35 μg/ml, 0.5 μg/ml to 25 μg/ml, 0.5 μg/ml to 20 μg/ml, 0.5 μg/ml to 15 μg/ml, 0.5 μg/ml to 10 μg/ml, or 0.5 μg/ml to 5 μg/ml, or 2 μg/ml to 8 μg/ml. Alternatively, the amount of clarithromycin may be in the range of 1 μg/ml to 75 μg/ml, 5 μg/ml to 75 μg/ml, 10 μg/ml to 75 μg/ml, 15 μg/ml to 75 μg/ml, 20 μg/ml to 75 μg/ml, 25 μg/ml to 75 μg/ml, 30 μg/ml to 75 μg/ml, 35 μg/ml to 75 μg/m, 40 μg/ml to 75 μg/m, 45 μg/ml to 75 μg/m, 50 μg/ml to 75 μg/m, 55 μg/ml to 75 μg/ml, or 60 μg/ml to 75 μg/ml. The amount of clarithromycin may also be 1 μg/ml, 2 μg/ml, 4 μg/ml, 6 μg/ml, 8 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 74.8 μg/ml or 75 μg/ml.

In some embodiments, the amount of rifaximin is in the range of 16 μg/ml to 64 μg/ml. For examples, the amount of rifaximin may be in the range of 16 μg/ml to 60 μg/ml, 16 μg/ml to 55 μg/ml, 16 μg/ml to 50 μg/ml, 16 μg/ml to 45 μg/ml, 16 μg/ml to 40 μg/ml, 16 μg/ml to 35 μg/ml, 16 μg/ml to 30 μg/ml, or 16 μg/ml to 25 μg/ml. Alternatively, the amount of clarithromycin may be in the range of 20 μg/ml to 64 μg/ml, 25 μg/ml to 64 μg/ml, 30 μg/ml to 64 μg/ml, 35 μg/ml to 64 μg/ml, 40 μg/ml to 64 μg/ml, 45 μg/ml to 64 μg/ml, or 50 μg/ml to 64 μg/ml. The amount of rifaximin may also be 19.6 μg/ml, 20 μg/m, 25 μg/m, 30 μg/m, 35 μg/m, 40 μg/m, 45 μg/m, 50 μg/m, 55 μg/m, 58.9 μg/mi or 60 μg/ml.

In some embodiments, the amount of rifaximin and clarithromycin each independently is in the range of 0.5 mg/kg to 50 mg/kg, for example, 0.5 mg/kg to 45 mg/kg, 0.5 mg/kg to 40 mg/kg, 0.5 mg/kg to 35 mg/kg, 0.5 mg/kg to 30 mg/kg, or 0.5 mg/kg to 25 mg/kg. Alternatively, the amount of rifaximin and clarithromycin each is independently in the range of 5 mg/kg to 50 mg/kg, 10 mg/kg to 50 mg/kg, 15 mg/kg to 50 mg/kg, 5 mg/kg to 20 mg/kg, or 20 mg/kg to 50 mg/kg. The amount of rifaximin and clarithromycin each independently may be 0.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or any amount between 0.5 to 50 mg/kg.

In some embodiments, for animal studies, such as in mice, the amount of rifaximin and clarithromycin each independently may be about 10 mg/kg.

It would be understood that the therapeutically effective amount for Rfx and Clr in combination will depend on various factors, such as the severity of the condition to be treated, the particular patient to be treated, the composition for a particular route of administration, as well as the compound(s) which is/are employed. In any event, the amount of a compound in the composition for a particular application may be determined routinely by the skilled person.

### C. Compositions

The antibiotic combination of the present invention may be provided in two forms: either as a single composition comprising both Clr and Rfx, or as separate compositions for concurrent administration, wherein one composition comprises Clr and the other composition comprises Rfx.

Thus, in some embodiments, the present invention provides a single composition comprising a therapeutically effective amount of Clr and Rfx and at least one pharmaceutically acceptable carrier, diluent or adjuvant. Preferably, said composition is administered by nasal, oral or topical route.

In some embodiments, the present invention provides a combination of two compositions, wherein one composition comprises Clr and at least one pharmaceutically acceptable carrier, and the other composition comprises Rfx and at least one pharmaceutically acceptable carrier, wherein the two compositions are each provided in a form that is suitable for administration in conjunction with the other and, together, they provide a therapeutic effect. In such embodiments, the two compositions may be administered by the same route or by different routes, depending on the disease to be treated. Preferably, the administration mode of each composition is independently selected from nasal, oral and topical administrations.

Preferably, such combination of two compositions is provided in the form of a kit to treat Mycobacterium infection. Accordingly, the present invention also provides a kit comprises the following components: (i) a composition comprising Clr and at least one pharmaceutically acceptable carrier; and (ii) a composition comprising Rfx and at least one pharmaceutically acceptable carrier, wherein components (i) and (ii) are each provided in a form that is suitable for administration in conjunction with the other.

Pharmaceutically acceptable carriers suitable for use in the present invention may be selected based on the chosen route of administration and standard pharmaceutical practice. Examples of carriers may include, but not limited to the following:
(a) a binder or a filler, which is selected from acacia, agar, alginic acid, carmellose sodium, dextrin, veegum or gel white, gellan gum, sodium alginate, hydroxypropyl starch, maltodextrin, modified starch, pectin, potassium alginate, polyvinyl pyrrolidone, carboxymethyl cellulose or an alkali metal salt thereof, microcrystalline cellulose, bentonite, col loidal silicon dioxide, microcrystalline cellulose/sodium carboxy methylcellulose, gum tragacanth, corn starch, or gelatin;
(b) a wetting agent, which is selected from alcohol, glycerin, propylene glycol, polyethylene glycol, mineral oil, benzalkonium chloride, benzethonium chloride, cetylpyridinium chloride, docusate sodium, nonoxynol 9, octoxynol, poloxamer, poloxamer 124, poloxamer 188, 237, 338, 407, polyoxyl 35 castor oil, polyoxyl 40;
(c) a suspending agent (sometimes also known as thickening agents), which is selected from gelatin, crosslinked polyacrylic acid, polymethacrylic acid, polyhydroxyethyl methacrylic acid, hydroxypropyl methyl cellulose, polyethylene glycol, sodium carboxymethyl cellulose, hyaluronic acid, chitosan, polycarbophil, pectin, copolymers of dextran, polyacrylamide, acacia, copolymer of caprolactone and ethylene oxide, carbopol 934, tragacanth, eudragit, polyvinyl pyrrolidone, polyacrylate and polyacrylate copolymer resins, celluloses and cellulose derivatives for example methyl-, ethyl- and propyl celluloses; hydroxyalkyl-celluloses, hydroxyl propyl celluloses, hydroxylpropylalkyl celluloses and the like including xanthan gum, polyvinyl resins, polyethylene glycol, polyethylene oxide, sorbitol, sucrose, xylitol, dextrose, fructose, maltitol, sugar, sodium alginate;
(d) a buffering agent, which is selected from acetate, amino acids, ammonium sulfate, benzoate, bicarbonate, borate, citrate, citric acid monohydrate, disodium hydrogen phosphate, glutamate, lactate, meglumine, potassium citrate, sodium acetate, sodium citrate, sodium phosphate, sulfate, tartrate, triethanolamine, TRIS, trisodium citrate dehydrate;
(e) a preservative, which is selected from benzyl alcohol, chloro-butanol, chloro-cresol, alkyl esters of paraben, phenol, phenyl ethanol, benzoic acid, potassium sorbate, sodium benzoate and antimicrobial solvents like propylene glycol, chloroform;
(f) a sweetening agent, which is selected from sucralose, sucrose, liquid glucose, glycerol, sorbitol, maltitol, saccharin sodium and aspartame;
(g) a flavouring agent, which is selected from essential oils including peppermint oil, orange oil, and lemon oil or can be selected from fruit flavour, e.g. peppermint flavour, strawberry flavour, tutti fruit flavour;
(h) colouring agents, including dyes and pigments such as Iron Oxide Red or Yellow, titanium dioxide, talc;
(i) pH control agents such as citric acid, tartaric acid, fumaric acid, sodium citrate, dibasic calcium phosphate, dibasic sodium phosphate; and
(j) surfactants or emulsifiers such as Pluronic, polyethylene glycols, sodium carboxymethyl cellulose, polyethoxylated and hydrogenated castor oil.

In some embodiments, the composition(s) described herein are suitable for oral administration. The composition may be in the dosage form of ingestible tablets, buccal tablets, powders, troches, pills, capsules, elixirs, suspensions, syrups, or wafers. In some embodiments, the composition(s) described herein are oral formulations of rifaximin and clarithromycin with diluents, binders, and other excipients for the treatment of NTM and other infections of the gastrointestinal tract.

In some embodiments, the composition(s) described herein are suitable for topical administration. For example, the composition may be in the dosage form of paints, liniments, creams, ointments, emulsions, pastes, plasters, lotions, patches, or gels.

In some embodiments, the composition(s) described herein are suitable for nasal administration. For example, the composition may be in the dosage form of nasal drops or sprays. For example, the composition may comprise micronized Clr or Rfx or both together with a suitable carrier such as lactose or other excipients, which composition is in the dosage form of dry powders suitable for aerosol delivery by nasal routes using, for example, a dry powder inhaler.

In some embodiments, the composition may consist of micronized rifaximin and carrier lactose or other excipients in portions that lead to a dry powder suitable for a dry powder inhaler. The clarithromycin may be administered by oral ingestion for distribution to the lung tissue to act in concert with the micronized rifaximin.

### D. Therapeutic Use

The present invention also provides the antibiotic combination, the composition or the kit described herein for use in the treatment of non-tuberculous mycobacteria (NTM) infections. The present invention also provides a use of the combination or the composition in the manufacture of a medicament for the treatment of NTM infections.

In some embodiments, the infection is a pulmonary infection, a gastrointestinal infection or a skin infection.

Preferably, the infection is caused by Mycobacterium abscessus and/or Mycobacterium avium. In such embodiments, the Mycobacterium abscessus and/or Mycobacterium avium is clarithromycin-resistant or non-resistant, preferably is clarithromycin-resistant. More preferably, the infection is caused by clarithromycin-resistant Mycobacterium abscessus.

The present invention also provides a method of treating mycobacterium infection in a subject, comprising administering a therapeutically effective combination of clarithromycin and rifaximin to said subject.

In some embodiments, the clarithromycin and the rifaximin are administered as a single composition. In other embodiments, the clarithromycin and the rifaximin are administered as separate compositions, which can be delivered simultaneously or sequentially. The definition of “composition” is as disclosed above in Section C.

In some embodiments, administration of the clarithromycin and the rifaximin results in synergistic inhibition of said mycobacterium infection.

It should be understood that any and all embodiments of the present disclosure can be combined with technical features in any other embodiment or multiple other embodiments to obtain additional embodiments under the premise of no conflict. The invention includes such combinations resulting in further embodiments.

### Examples

The examples and exemplary embodiments below are intended to be purely exemplary of the invention and should therefore not be considered to limit the invention in any way.

### Materials and Methods

### Bacterial Strains and Culture Media

Mycobacterium abscessus strain M422 was used for screening and hit confirmation since it exhibits robust inducible resistance against Clr. For checkerboard synergy assay, 3 M. abscessus strains were used: M. abscessus ATCC 19977, M. abscessus strains M422 and M110, while bactericidal activity determination and the zebrafish study were performed with M. abscessus ATCC 19977. All three strains of M. abscessus harbor the inducible clarithromycin resistance-conferring erm(41) T28 sequevar. M. abscessus strain M422 and M110 were provided by Dr. Jeanette Teo, National University Hospital, Singapore (Chew K L, et al., 2017. J Med Microbiol 66: 1443-1447). M. abscessus ATCC 19977 and M. avium ATCC 700898 were acquired from American Type Culture Collection (ATCC).

To visualize the bacterial infection in the zebrafish (Denio rerio), a fluorescent strain of M. abscessus ATCC 19977 containing pJKD2893:mScarlet was generated. The pJKD2893 μlasmid backbone was a kind gift from Professor Timothy Stinear. The gene expressing the fluorescent protein mScarlet was amplified from μMRE135-mScarlet (Addgene #118489, Mitja Remus-Emsermann lab). The amplification product was annealed by Gibson cloning with the pJKD2893 backbone amplified from a sample of pJKD2893. The resulting plasmid, termed pJKD2893:mScarlet, was electroporated into M. abscessus ATCC 19977 at a culture density equivalent to OD₆₀₀˜20 using a BioRad GenePulser Xcell set at 2.5 kV, 25 uF, and 1,000Ω. After electroporation, the culture was expanded to 2-5 ml and left to recover at 32° C. for 24 hours in complete 7H9 media without selection drug. The culture was subsequently concentrated to 100 μl and plated on 7H11 supplemented with 100 μg/ml kanamycin. Colonies carrying the plasmid were identified by their fluorescence.

All liquid bacterial cultures were grown in Middlebrook 7H9 broth (BD Difco) supplemented with 0.5% albumin, 0.5% glycerol, 0.2% glucose, 0.085% sodium chloride, and 0.05% Tween 80. Solid cultures were grown on Middlebrook 7H10 agar (BD Difco) supplemented with 0.5% albumin, 0.5% glycerol, 0.2% Dextrose, 0.085% sodium chloride, 0.006% oleic acid, and 0.0003% catalase.

### Primary Compound Screening Against Pre-Induced Bacterial Cells

A total of 2,252 drugs/compounds comprising 1,576 FDA-approved drugs and 676 clinical compounds were purchased from MedChemExpress. The compounds were dissolved in dimethyl sulfoxide (DMSO) to a stock concentration of 10 mM. The Clr resistance in M. abscessus was induced for 3 days under 0.5 μg/ml of Clr before being subjected to compound screening. M. abscessus strain M422 was selected for the primary screening as it exhibited robust inducible Clr resistance. After 3 days of incubation in an airtight container with moist paper towels at 37° C., the MIC of Clr increased from 4 μg/ml to 128 μg/ml.

Primary screening was carried out in 96-well flat-bottom Corning Costar cell culture plates at 50 μM compound along with 4 μg/ml of Clr in a starting inoculum of pre-induced M. abscessus cells at OD₆₀₀ of 0.005 in a final volume of 100 μl. The plates were put in an airtight container with moist paper towels and incubated for 4 d at 30° C. The cultures in the wells were resuspended before OD₆₀₀ was read in a BioTek Synergy 4 μlate reader. Compounds were defined as hits if they showed growth inhibition of >90% compared to the untreated control.

### Checkerboard Synergy Assay

With Rfx identified as the best potentiator of Clr activity, the interaction between Rfx and Clr was investigated by a checkerboard synergy approach using a broth microdilution method performed in 96-well plates. To provide a classification of the combined antibiotics based on the fractional inhibitory concentration index (FICI), this assay applied the combination of Clr and Rfx in concentrations of 0-64 μg/ml by 2-fold serial dilutions along the abscissa and ordinate, respectively. The FICI was calculated by summing the FIC of Rfx and the FIC of Clr. FIC of Clr is the MIC of Clr in the presence of Rfx divided by MIC of Clr alone. FIC of Rfx was similarly calculated. The drug interactions between Rfx and Clr were defined as synergistic when they had a FICI of <0.5.

### Bactericidal/Static Activity Determination

To determine bactericidal and bacteriostatic activity, a series of MBC assays were performed on M. abscessus ATCC 19977. From the checkerboard synergy assay plate, the well with the most synergistic combination of Clr and Rfx (1×MIC) was determined. The wells corresponding to the 5 concentrations (0.5×, 1×, 2×, 4×, 8×MIC) of Rfx in the presence of 1×MIC Clr were plated. Similarly, the wells that correspond to the 5 concentrations (0.5×, 1×, 2×, 4×, 8×MIC) of Clr in the presence of 1×MIC Rfx were plated. Serial 10-fold dilutions were performed on the above-mentioned combinations of Clr and Rfx. Aliquots (50 μl) of each dilution was spread onto 7H10 agar plates, which were incubated at 37° C. for 3 days and the CFU enumerated. MBC₉₀ is defined as the lowest drug concentration required to induce 90% cell death as compared to the untreated control at 0 h time point. The combination of Clr and Rfx is defined as bactericidal if its MBC₉₀ is ≤4×MIC₉₀.

### Zebrafish Strains, Breeding, and Housing

All work with zebrafish was approved by the Nanyang Technological University Institutional Animal Care and Use Committee (Animal Use Protocol #A20038). Adult zebrafish were housed in a facility kept at 28.5° C. and light/dark cycles of 14 h/10 h. Only wildtype zebrafish of the AB strain were used for these experiments.

Preparation of M. abscessus Inoculum and Zebrafish Infections Cultures of M. abscessus-pJKD2893:mScarlet in 7H9 media supplemented with 50 μg/ml kanamycin were grown to OD₆₀₀ of 0.2-0.3 and concentrated to OD₆₀₀ of 1 in 0.1% PBS-Tween-20. The inoculum was prepared by homogenizing the bacterial suspension through a 26-gauge needle and subjecting it to ultrasonication at low power, as described by Bernut et al. (Bernut et al., Antimicrob Agents Chemother 58: 4054-4063). The homogenate was left for 10 min for clumps to settle and the supernatant was subsequently moved to a new tube and mixed with phenol red to a final concentration of 0.1%. Larvae aged 48 h post-fertilization were dechorionated and anaesthetized by immersion with MS-222 (Sigma-Aldrich). M. abscessus-pJKD2893:mScarlet in 0.1% PBS-Tween-20 and 20% phenol red (2-3 nl containing 300-350 CFU) was injected into the hindbrain ventricle using an Eppendorf FemtoJet microinjector. The actual CFU in the inoculum was determined a posteriori by injecting into 50 μl 0.1% PBS-Tween-20 and streaking on a plate. Infected larvae were kept in E3 medium for 24 h, whereupon they were randomly divided into groups for antibiotic treatments. The larvae were treated by immersion in water containing the antibiotics and the water was changed daily.

### Bacterial Recovery and CFU Determination

Larvae were harvested to assess the bacterial load at 5 d post-infection using a slightly modified version of the Kremer lab method (Bernut et al., Antimicrob Agents Chemother 58: 4054-4063). Briefly, groups of five larvae were transferred to a 1.5 ml microcentrifuge tube and washed twice with 0.1% PBS-Tween-20. The larvae were homogenized together in 100 μl 0.3% PBS-Triton-X using a hand-held motorized homogenizer with 1.5 ml pestle. The homogenate was run through a 26-gauge syringe, diluted, and spread on 7H11-agar containing 100 μg/ml kanamycin, 250 μg/ml amphotericin B, and 25 μg/ml hygromycin. Fluorescent colonies were counted after 5-6 days.

### Statistical Analyses

Data were analyzed by using two-tailed Student's t-test with Welch's correction, and a value of P<0.05 was considered statistically significant (*P<0.05; **P<0.01).

### Results

Identification of Rifaximin as a Clarithromycin Potentiator in M. abscessus.

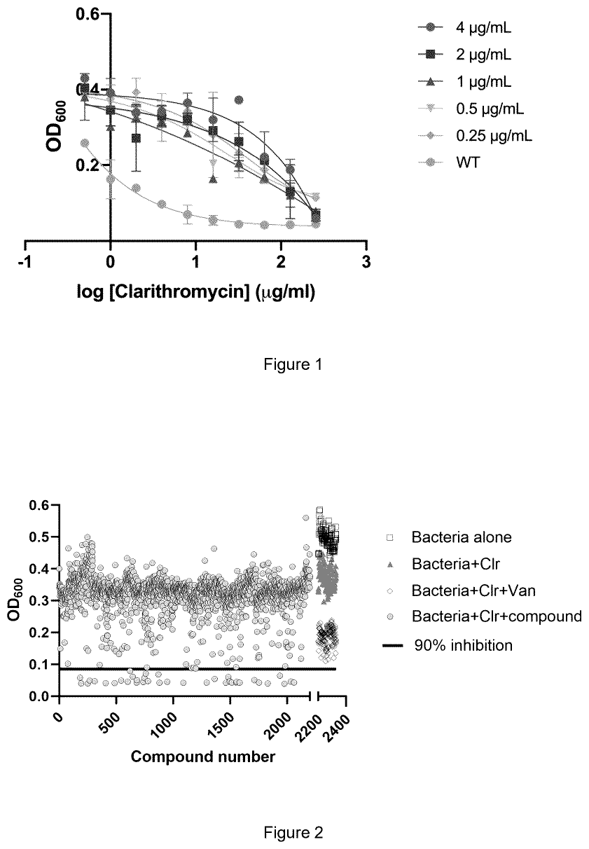

The minimum inhibitory concentration (MIC) of M. abscessus clinical strain M422 before and after 3 days of induction was detected to ensure successful induction of Clr resistance. As shown in FIG. 1, the MIC of uninduced bacterial cell (WT) is 4-8 μg/ml, whereas the cells exhibited MIC values above 64 μg/ml once they were induced with clarithromycin concentrations of 0.25-4 μg/ml for 3 days. A sub-inhibitory concentration of 0.5 μg/ml was then selected for the following screen.

A compound library consisting of 2,252 FDA-approved and clinical-stage drugs was screened against M. abscessus clinical strain M422. Bacteria were exposed to a sub-inhibitory concentration of Clr (0.5 μg/ml) for 3 days to induce Clr resistance. The test drugs were screened at 50 μM in the presence of 4 μg/ml of Clr to maintain the Clr-resistance phenotype. FIG. 2 shows the scatter plot of the screen.

From the 2,252 compounds, only 38 showed >90% growth inhibition. To start the validation process, the top 10 compounds with >90% growth inhibition were chose to perform a single-concentration assay, with and without clarithromycin. Finally, the inventors obtained 7 compounds that potentiated Clr efficacy. These 7 compounds are listed in Table 1.

TABLE 1
Compounds showing potentiated activity when
combining with clarithromycin (Clr)

| | % inhibition with 0.5 | % inhibition with |
|---|---|---|
| Compounds | μg/ml Clr | compound alone |
| Rifaximin | 100 | 85.6 |
| Novobiocin | 99.7 | 84.1 |
| (Sodium) | | |
| Tedizolid | 99.1 | 85.2 |
| Cefotaxime (sodium | 98.5 | 86.7 |
| salt) | | |
| AN-2690 | 96.7 | 80.4 |
| Sutezolid | 92.8 | 80.4 |
| Auranofin | 98.5 | 86.9 |

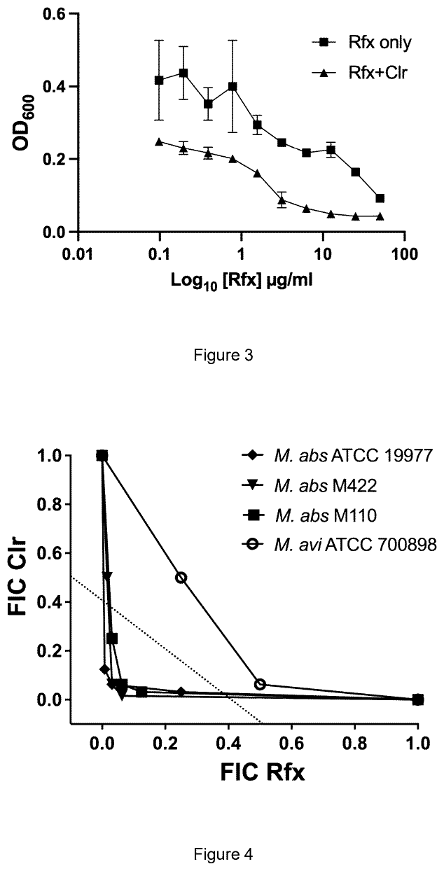

After performing dose-response and checkerboard synergy assays on the 7 compounds, the inventors found rifaximin to be the only compound that showed a strong synergy profile with a pharmacologically-relevant effective concentration needed to achieve >99% killing (Sutezolid did not show significant synergistic activity with Clr, while very high concentrations were needed for Novobiocin, Tedizolid, Cefotaxime, AN-2690, and Auranofin to show synergistic effects with Clr). Additionally, the dose response of rifaximin on strain M422 with and without the presence of 4 μg/ml of Clr was detected, and the MIC of Rfx was significantly lowered with the presence of Clr (FIG. 3).

### The Interaction Between Cr and Rfx is Synergistic and Bactericidal

To characterize the interaction between Clr and Rfx, checkerboard synergy assays were performed on pre-induced Clr-resistant M. abscessus. The fractional inhibitory concentration index (FICI) obtained from the checkerboard assay defines whether the interaction between the two compounds is either synergistic (FICI≤0.5), additive (0.5<FICI≤1.0), indifferent (1.0<FICI≤4.0), or antagonistic (FICI >4.0) (Noel D J, Keevil C W, Wilks S A. 2021. MBio 12). As shown in Table 2 and FIG. 4, the combination of Clr and Rfx exhibits strong synergy (FICI <0.1) in all 3 M. abscessus strains tested. Importantly, the MIC of Clr was reduced by at least 16-fold compared to clinically relevant concentrations (2-8 μg/ml) when used in in combination with Rfx (FIG. 4, Table 2).

TABLE 2
Checkerboard synergy assay for Clr and Rfx against
M. abscessus and M. avium strains

| | MIC₉₀ (μg/ml) | | | | |
|---|---|---|---|---|---|
| | individually | | in combination | | |
| Strain | Clr | Rfx | Clr | Rfx | FICI |
| M. abscessus ATCC 19977 | 128 | 128 | 8 | 4 | 0.094 |
| (pre-induced) | | | | | |
| M. abscessus strain M422 | 64 | 16 | 2 | 1 | 0.094 |
| (pre-induced) | | | | | |
| M. abscessus strain M110 | 128 | 128 | 2 | 4 | 0.047 |
| (pre-induced) | | | | | |
| M. avium ATCC 700898 | 0.5 | 0.03 | 0.031 | 0.015 | 0.563 |
| (uninduced) | | | | | |

As both M. abscessus and M. avium are the main organism groups causing pulmonary NTM (Lim A Y H, et al., 2018. BMC Pulm Med 18 (1):85; Tan Y, et al., 2018. BMC Pulm Med 18 (1): 168), the inventors tested the combination of Clr and Rfx against M. avium in the checkerboard assay as well. With a FICI of 0.563, the interaction between Clr and Rfx was additive in M. avium. However, given that Cir and Rfx are highly potent in M. avium (MIC₉₀<0.1 μg/ml), the antibiotic combination could still be effective to treat lung infections caused by M. avium.

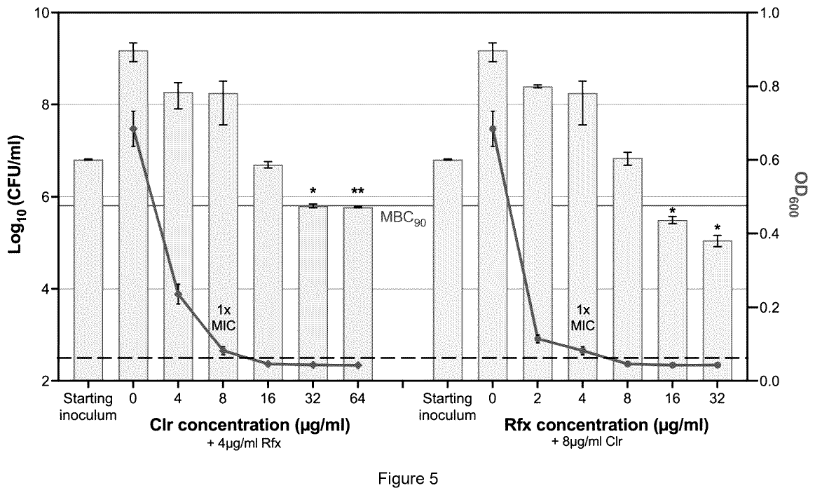

To determine whether the combination of Clr and Rfx is bactericidal, M. abscessus ATCC 19977 was treated with the antibiotics at 0.5×, 1×, 2×, 4×, 8×MICs in the presence of 1×MIC of the partner antibiotic, and bacterial viability was determined by CFU enumeration on agar plates. Minimum bactericidal concentration (MBCo₉₀) was defined as the lowest drug concentration required to induce >90% cell death as compared to the starting inoculum (at 0 h time point) of the untreated control. The number of residual log₁₀ CFU/mL was determined through plating 10-fold serial dilution and compared with the starting inoculum. FIG. 5 shows that MBC₉₀ was achieved with either Clr 32 μg/ml+Rfx 4 μg/ml or Clr 8 μg/ml+Rfx 16 μg/ml, indicating that combining these two antibiotics result in a bactericidal combination.

### The Combination Cr and Rfx is Efficacious in a Zebrafish Embryo Infection Model.

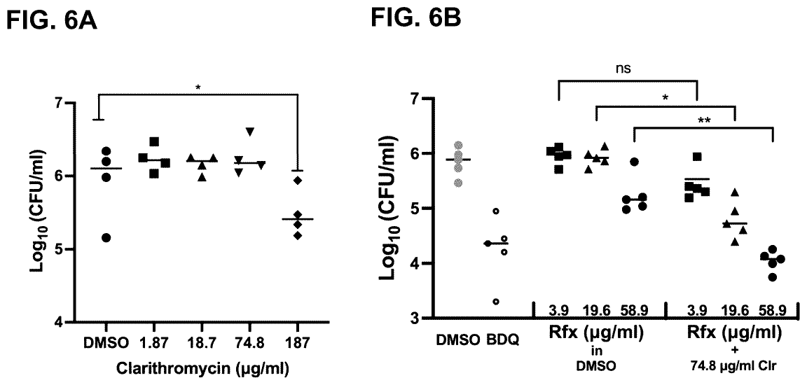

Given the strong synergy observed in vitro, the efficacy of the Clr+Rfx combination was evaluated in vivo. The zebrafish embryo infection model was used in this study as it is a well-characterized in vivo model system for compound testing against M. abscessus (Bernut A, et al., 2014. Antimicrob Agents Chemother 58: 4054-4063). First, the potency of Clr was evaluated to determine a sub-inhibitory concentration that could be used in subsequent combination experiments. Specifically, fish were infected in the hindbrain ventricle at 48 hours post fertilization (hpf), and treatment began at 72 hpf with 4 different concentrations of Clr ranging from 1.87 μg/ml to 187 μg/ml (corresponding to 2.5-250 μM). Treatment was maintained for 4 days with daily water changes before the fish were assessed for bacterial burden. The result shows that Clr was unable to reduce bacterial load up to a concentration of 74.8 μg/ml (corresponding to 100 μM) (FIG. 6A). This concentration was selected as the sub-inhibitory concentration of Clr to evaluate its interaction with Rfx in the zebrafish infection model.

Then, infected fish at 72 hpf were treated with different concentrations of Rfx from 3.9 μg/ml (corresponding to 5 μM) to 58.9 μg/ml (corresponding to 75 μM) with or without the presence of 74.8 μg/ml Clr. 3 μg/ml Bedaquiline (BDQ) was used as a positive control as it has demonstrated efficacy against M. abscessus in zebrafish embryos. DMSO was used as a negative control. The combination of 19.6 μg/ml (corresponding to 25 μM) of Rfx and 74.8 μg/ml of Clr resulted in >1 log CFU reduction, while 19.6 μg/ml of Rfx alone did not show any CFU reduction (FIG. 6B). Similarly, 58.9 μg/ml (75 μM) of Rfx and 74.8 μg/ml of Clr reduced the bacterial load by >2 log in CFU count compared to Rfx treatment alone, showing that the combination is potent in vivo (FIG. 6B).

### The Combination Cr and Rfx is Superior Over the Known Antibiotic Combinations Against XX

The effects of known antibiotic combinations of clarithromycin with each of tigecycline, ciprofloxacin, moxifloxacin, rifabutin, vancomycin, imipenem and oritavancin diphosphate were detected by checkerboard synergy assay using pre-induced Clr-resistant M. abscessus ATCC 19977 strain. Results are shown in Tables 3-9. Data highlighted in grey indicates cells having less than 10% growth.

TABLE 3
Percentage of bacterial growth under combinations of different
concentrations of clarithromycin and tigecycline.

| | | | | | | | | | |
|---|---|---|---|---|---|---|---|---|---|
| Clarithro- | 64 | 43.0 | 43.7 | 37.0 | 33.0 | 22.3 | 9.0 | 1.5 | 0.3 |
| mycin | 32 | 77.4 | 68.9 | 56.2 | 35.4 | 29.6 | 10.2 | 2.9 | 3.5 |
| (μg/ml) | 16 | 64.0 | 56.5 | 49.6 | 38.8 | 27.2 | 9.5 | 1.5 | 0.4 |
| | 8 | 76.2 | 67.2 | 63.1 | 42.6 | 27.8 | 10.3 | 3.2 | 0.4 |
| | 4 | 86.2 | 68.7 | 58.4 | 50.6 | 32.0 | 15.2 | 6.3 | 1.5 |
| | 2 | 80.6 | 76.4 | 69.5 | 57.7 | 35.0 | 18.1 | 8.3 | 0.3 |
| | 1 | 80.1 | 74.9 | 68.5 | 57.9 | 46.5 | 16.7 | 7.8 | 1.9 |
| | 0 | 100.0 | 64.2 | 74.2 | 77.8 | 67.0 | 39.3 | 22.4 | 10.1 |
| | | 0 | 1 | 2 | 4 | 8 | 16 | 32 | 64 |

Tigecycline (μg/ml)
FICI = 0.25

TABLE 4
Percentage of bacterial growth under combinations of different
concentrations of clarithromycin and ciprofloxacin.

| | | | | | | | | | |
|---|---|---|---|---|---|---|---|---|---|
| Clarithro- | 64 | 95.2 | 71.5 | 47.4 | 16.5 | 8.1 | 3.3 | 1.8 | 0.5 |
| mycin | 32 | 84.3 | 85.9 | 58.3 | 17.8 | 7.7 | 4.2 | 2.2 | 0.6 |
| (μg/ml) | 16 | 109.5 | 91.9 | 55.8 | 17.7 | 9.9 | 4.8 | 2.7 | 1.0 |
| | 8 | 102.8 | 115.4 | 70.3 | 26.2 | 11.9 | 7.3 | 3.4 | 1.5 |
| | 4 | 113.2 | 112.8 | 96.2 | 38.8 | 14.2 | 8.8 | 2.6 | 1.3 |
| | 2 | 99.5 | 123.0 | 100.5 | 81.1 | 19.7 | 8.5 | 3.4 | 1.3 |
| | 1 | 116.9 | 101.5 | 126.6 | 104.7 | 26.6 | 11.7 | 3.6 | 1.2 |
| | 0 | 100.0 | 112.4 | 113.7 | 112.2 | 50.7 | 9.9 | 3.8 | 0.7 |
| | | 0 | 1 | 2 | 4 | 8 | 16 | 32 | 64 |

Ciprofloxacin (μg/ml)
FICI > 0.5

TABLE 5
Percentage of bacterial growth under combinations of different
concentrations of clarithromycin and moxifloxacin.

| | | | | | | | | | |
|---|---|---|---|---|---|---|---|---|---|
| Clarithro- | 64 | 65.0 | 56.8 | 33.7 | 20.4 | 14.0 | 12.6 | 5.5 | 0.7 |
| mycin | 32 | 83.8 | 66.2 | 37.4 | 21.3 | 15.0 | 10.6 | 8.0 | 0.7 |
| (μg/ml) | 16 | 86.1 | 67.1 | 39.4 | 23.4 | 13.7 | 13.8 | 10.0 | 0.5 |
| | 8 | 93.2 | 82.1 | 52.5 | 25.9 | 14.9 | 11.5 | 6.8 | 0.7 |
| | 4 | 103.4 | 105.5 | 74.7 | 33.3 | 16.2 | 10.1 | 7.8 | 0.8 |
| | 2 | 102.2 | 111.2 | 94.0 | 48.0 | 20.2 | 9.4 | 7.0 | 0.6 |
| | 1 | 119.5 | 109.8 | 97.2 | 81.4 | 29.8 | 10.4 | 6.4 | 0.7 |
| | 0 | 100.0 | 107.8 | 122.8 | 86.4 | 51.2 | 13.4 | 7.4 | 0.6 |
| | | 0 | 1 | 2 | 4 | 8 | 16 | 32 | 64 |

Moxifloxacin (μg/ml)
FICI > 0.5

TABLE 6
Percentage of bacterial growth under combinations of different
concentrations of clarithromycin and rifabutin.

| | | | | | | | | | |
|---|---|---|---|---|---|---|---|---|---|
| Clarithro- | 64 | 99.8 | 97.8 | 91.2 | 87.2 | 55.1 | 10.6 | 1.7 | 1.6 |
| mycin | 32 | 115.7 | 116.4 | 97.1 | 91.6 | 60.9 | 17.9 | 2.2 | 1.9 |
| (μg/ml) | 16 | 101.8 | 105.8 | 89.4 | 90.3 | 62.1 | 30.5 | 2.0 | 1.7 |
| | 8 | 108.1 | 105.3 | 98.3 | 99.9 | 70.3 | 35.4 | 4.4 | 1.5 |
| | 4 | 117.2 | 114.4 | 116.0 | 113.4 | 85.6 | 43.8 | 4.7 | 1.6 |
| | 2 | 98.9 | 104.2 | 111.0 | 106.6 | 99.6 | 61.8 | 3.4 | 1.8 |
| | 1 | 108.3 | 123.4 | 114.2 | 115.5 | 104.5 | 68.7 | 6.0 | 9.5 |
| | 0 | 100.0 | 105.5 | 101.8 | 99.4 | 100.2 | 79.1 | 9.2 | 1.2 |
| | | 0 | 1 | 2 | 4 | 8 | 16 | 32 | 64 |

Rifabutin (μg/ml)
FICI = 1

TABLE 7
Percentage of bacterial growth under combinations of different
concentrations of clarithromycin and vancomycin.

| | | | | | | | | | |
|---|---|---|---|---|---|---|---|---|---|
| Clarithro- | 64 | 60.5 | 60.1 | 60.2 | 58.3 | 60.6 | 56.3 | 55.7 | 55.8 |
| mycin | 32 | 59.0 | 57.8 | 62.3 | 61.8 | 61.4 | 56.9 | 58.3 | 69.3 |
| (μg/ml) | 16 | 72.3 | 66.8 | 70.5 | 68.7 | 68.7 | 66.3 | 72.8 | 63.7 |
| | 8 | 71.4 | 72.2 | 67.6 | 69.4 | 70.0 | 71.4 | 73.6 | 73.1 |
| | 4 | 82.7 | 84.2 | 82.7 | 84.0 | 83.3 | 81.2 | 82.6 | 82.8 |
| | 2 | 80.2 | 79.5 | 80.5 | 80.0 | 81.9 | 86.5 | 89.3 | 91.6 |
| | 1 | 97.8 | 100.4 | 94.0 | 102.3 | 98.2 | 99.2 | 103.0 | 99.3 |
| | 0 | 100.0 | 103.7 | 102.0 | 105.7 | 91.7 | 90.8 | 86.6 | 83.1 |
| | | 0 | 1 | 2 | 4 | 8 | 16 | 32 | 64 |

Vancomycin (μg/ml)

TABLE 8
Percentage of bacterial growth under combinations of different
concentrations of clarithromycin and imipenem.

| | | | | | | | | | |
|---|---|---|---|---|---|---|---|---|---|
| Clarithro- | 64 | 93.7 | 78.8 | 116.5 | 38.0 | 55.3 | 39.7 | 38.2 | 23.8 |
| mycin | 32 | 98.2 | 82.4 | 77.4 | 61.9 | 51.1 | 43.0 | 29.6 | 22.9 |
| (μg/ml) | 16 | 89.6 | 95.2 | 89.6 | 39.4 | 61.0 | 38.9 | 27.6 | 25.2 |
| | 8 | 97.4 | 91.9 | 94.4 | 45.2 | 64.1 | 53.1 | 34.4 | 25.0 |
| | 4 | 78.4 | 89.3 | 80.1 | 40.0 | 71.5 | 52.1 | 35.4 | 21.3 |
| | 2 | 84.8 | 95.6 | 83.0 | 46.4 | 74.1 | 57.3 | 39.5 | 20.0 |
| | 1 | 90.2 | 59.8 | 95.6 | 49.8 | 80.4 | 58.6 | 42.7 | 25.6 |
| | 0 | 100.0 | 86.9 | 93.4 | 58.5 | 78.5 | 62.1 | 38.3 | 16.6 |
| | | 0 | 1 | 2 | 4 | 8 | 16 | 32 | 64 |

Imipenem (μg/ml)

TABLE 9
Percentage of bacterial growth under combinations of different
concentrations of clarithromycin and oritavancin diphosphate.

| | | | | | | | | | |
|---|---|---|---|---|---|---|---|---|---|
| Clarithro- | 64 | 85.8 | 56.1 | 50.2 | 42.4 | 39.5 | 36.5 | 29.9 | 22.6 |
| mycin | 32 | 95.4 | 57.3 | 50.5 | 43.0 | 40.5 | 35.6 | 29.8 | 23.7 |
| (μg/ml) | 16 | 89.7 | 54.3 | 48.0 | 41.1 | 40.3 | 33.8 | 26.9 | 21.8 |
| | 8 | 92.9 | 55.4 | 50.0 | 43.7 | 42.5 | 35.2 | 27.4 | 21.9 |
| | 4 | 93.3 | 56.7 | 49.9 | 45.4 | 46.1 | 38.7 | 27.9 | 21.8 |
| | 2 | 96.8 | 60.8 | 53.0 | 49.4 | 45.8 | 38.7 | 29.9 | 23.5 |
| | 1 | 95.5 | 59.9 | 58.9 | 51.8 | 46.3 | 37.7 | 28.3 | 22.4 |
| | 0 | 100.0 | 68.0 | 59.1 | 51.1 | 46.8 | 40.9 | 32.2 | 26.1 |
| | | 0 | 1 | 2 | 4 | 8 | 16 | 32 | 64 |

Oritavancin diphosphate (μg/ml)

Among the tested 7 combinations, tigecycline, ciprofloxacin, rifabutin and moxifloxacin each combined with clarithromycin showed growth inhibition between 8 to 64 μg/ml, while the other 3 combinations showed no significant growth inhibition even at a high concentration of 64 μg/ml.

It is further noted that tigecycline has an FICI of 0.25, indicating its synergistic effects with clarithromycin. However, tigecycline only exhibited such synergistic effects at a concentration of 16 μg/ml and above (Table 3). To the contrary, rifaximin is able to show significant synergistic effects with clarithromycin at a concentration as low as 1 μg/ml (Table 2). Thus, the combination of rifaximin and clarithromycin of the present disclosure is superior over the known combination of tigecycline and clarithromycin.

### SUMMARY

NTM treatment routinely involves the use of several antibiotics to enhance efficacy and to minimize the development of resistance. As the cornerstone of M. abscessus treatment, Clr is often included in the combination therapies based on anecdotal or clinical experience. The identification of synergistic combinations, however, can be achieved more efficiently by screening combinations of approved drugs in vitro, which could accelerate bench-to-bedside translation since the approved drugs have previously been evaluated for safety. Using this approach, the inventors identified Rfx and showed that it acts synergistically with Clr against M. abscessus both in vitro and in vivo.

Rfx was overlooked in previous M. abscessus screening campaigns because M. abscessus contains ADP-ribosyltransferase that accounts for the inactivation and resistance to several rifamycin derivatives including rifaximin (Baysarowich J, et al., 2008. Proc Natl Acad Sci USA 105: 4886-4891; Rominski A, et al., 2017. J Antimicrob Chemother 72: 376-384). Indeed, the inventors found the MIC of Rfx is extremely high when tested alone. However, since the inventors set out to specifically search for Clr potentiators by performing compound screening in presence of Clr, this potent combination of Rfx and Clr was successfully identified.

Among the rifamycin family, rifabutin was reported in several independent studies to exhibit bactericidal activity against M. abscessus and display synergy with Clr (Aziz D B, et al., 2017. Antimicrob Agents Chemother 61(6): e00155-17). Unsurprisingly then, rifabutin was also identified as one of the top hits in the primary screening of the present disclosure. However, rifabutin failed to exhibit synergistic effects with Clr in the checkerboard synergy assay using pre-induced Clr-resistant M. abscessus strain ATCC19977 (Table 6). Given that Rfx and rifabutin are from the same family, Rfx probably inhibits the induction of erm(41) gene by targeting its transcription, thereby enabling Clr to remain effective (Ganapathy U S, Dartois V, Dick T. 2019. Expert Opin Drug Discov 14: 867-878).

Rfx was approved to treat gastrointestinal infections only because of its low oral bioavailability. The observation of the Clr+Rfx synergy may allow us to leverage the non-systemic nature of Rfx to treat M. abscessus lung infections by administering Rfx by aerosol delivery. An aerosolized form of Rfx was used to treat Pseudomonas aeruginosa lung infection in mice (Kirby B D, et al., 2019. Antimicrob Agents Chemother 63 (7):e02341-18), indicating that this approach could be applied to NTM infections. Pulmonary infection with M. abscessus is primarily an extracellular infection of the sputum where the bacteria mainly reside on the epithelia surface of the lung and sputum. It is challenging for an antibiotic administered orally to achieve a high concentration at the interstitial space of lung tissue and sputum in the alveolar. This limitation can be overcome by aerosol administration that enables drugs of desired concentrations to be loaded directly into the lungs. Hence, the proposed approach of nasal co-administration of Rfx and Clr is a feasible therapeutic option for pulmonary M. abscessus infection.

For one skilled in the art, various modifications and changes may be made to the present disclosure. Those skilled in the art should understand that any amendments, equivalent replacements, improvements, and so on, made within the spirit and principle of the present disclosure, should be covered within the scope of protection of the present disclosure.

### BIBLIOGRAPHY

1. Chew K L, Cheng J W S, Hudaa Osman N, Lin R T P, Teo J W P. Predominance of clarithromycin-susceptible Mycobacterium massiliense subspecies: Characterization of the Mycobacterium abscessus complex at a tertiary acute care hospital. J Med Microbiol. 2017 October; 66(10): 1443-1447.
2. Lim A Y H, Chotirmall S H, Fok E T K, Verma A, De P P, Goh S K, Puah S H, Goh D E L, Abisheganaden J A. Profiling non-tuberculous mycobacteria in an Asian setting: characteristics and clinical outcomes of hospitalized patients in Singapore. BMC Pulm Med. 2018 May 22; 18(1): 85.
3. Tan Y, Su B, Shu W, Cai X, Kuang S, Kuang H, Liu J, Pang Y. Epidemiology of pulmonary disease due to nontuberculous mycobacteria in Southern China, 2013-2016. BMC Pulm Med. 2018 Nov. 9; 18(1): 168.
4. Bernut A, Le Moigne V, Lesne T, Lutfalla G, Herrmann J L, Kremer L. In vivo assessment of drug efficacy against Mycobacterium abscessus using the embryonic zebrafish test system. Antimicrob Agents Chemother. 2014 July; 58(7): 4054-63.
5. Baysarowich J, Koteva K, Hughes D W, Ejim L, Griffiths E, Zhang K, Junop M, Wright G D. Rifamycin antibiotic resistance by ADP-ribosylation: Structure and diversity of Arr. Proc Natl Acad Sci USA. 2008 Mar. 25; 105(12): 4886-91.
6. Rominski A, Roditscheff A, Selchow P, Böttger EC, Sander P. Intrinsic rifamycin resistance of Mycobacterium abscessus is mediated by ADP-ribosyltransferase MAB_0591. J Antimicrob Chemother. 2017 February; 72(2): 376-384.
7. Aziz D B, Low J L, Wu M L, Gengenbacher M, Teo J W P, Dartois V, Dick T. Rifabutin Is Active against Mycobacterium abscessus Complex. Antimicrob Agents Chemother. 2017 May 24; 61(6): e00155-17.
8. Ganapathy U S, Dartois V, Dick T. Repositioning rifamycins for Mycobacterium abscessus lung disease. Expert Opin Drug Discov. 2019 September; 14(9): 867-878.
9. Kirby B D, Al Ahmar R, Withers T R, Valentine M E, Valentovic M, Long T E, Gaskins J R, Yu H D. Efficacy of Aerosolized Rifaximin versus Tobramycin for Treatment of Pseudomonas aeruginosa Pneumonia in Mice. Antimicrob Agents Chemother. 2019 Jun. 24; 63(7): e02341-18.

1. An antibiotic combination comprising a therapeutically effective amount of clarithromycin and rifaximin, wherein the combination is bactericidal to clarithromycin-resistant and clarithromycin non-resistant mycobacteria.

2. The antibiotic combination according to claim 1, wherein the mycobacterium is Mycobacterium abscessus or Mycobacterium avium.

3. The antibiotic combination according to claim 2, wherein the combination has a fractional inhibitory concentration index (FICI) ≤0.5 for Mycobacterium abscessus.

4. The antibiotic combination according to claim 1, wherein the clarithromycin and the rifaximin are formulated as a single composition or as separate compositions, said separate compositions are to be administered in conjunction, simultaneously or sequentially.

5. The antibiotic combination according to claim 1, wherein the composition further comprises a pharmaceutically acceptable carrier and/or excipient.

6. The antibiotic combination according to claim 1, wherein the composition is for administration by nasal, oral or topical routes.

7. The antibiotic combination according to claim 1, wherein the therapeutically effective amount comprises:
i) clarithromycin in the range of 0.5 μg/ml to 75 μg/ml, and rifaximin in the range of 1 μg/ml to 64 μg/ml, or
ii) rifaximin and clarithromycin each independently in the range of 0.5 mg/kg to 50 mg/kg, preferably in the range of 5 mg/kg to 20 mg/kg, more preferably at 10 mg/kg.

8. The antibiotic combination according to claim 1 for use in the treatment of non-tuberculous mycobacteria (NTM) infections.

9. (canceled)

10. (canceled)

11. (canceled)

12. (canceled)

13. (canceled)

14. A method of treating Mycobacterium infections in a subject, comprising administering a therapeutically effective combination of clarithromycin and rifaximin of claim 1 to said subject.

15. The method according to claim 14, wherein the clarithromycin and the rifaximin are administered as a single composition.

16. The method according to claim 14, wherein the clarithromycin and the rifaximin are administered as separate compositions.

17. The method according to claim 14, wherein the infection is a pulmonary infection, a gastrointestinal infection or a skin infection.

18. The method according to claim 14, wherein the infection is caused by Mycobacterium abscessus and/or Mycobacterium avium.

19. The method according to claim 14, wherein administration of the clarithromycin and the rifaximin results in synergistic inhibition of said Mycobacterium infection.

20. A kit to treat Mycobacterium infection, comprising the antibiotic combination according to claim 1.

21. The kit of claim 20, wherein the infection is a pulmonary infection, a gastrointestinal infection or a skin infection.

22. The kit of claim 20, wherein the infection is caused by;
i) Mycobacteriumm abscessus and/or Mycobacterium avium; or
ii) clarithromycin-resistant Mycobacterium abscessus.

23. The kit according to claim 20, wherein the kit comprises the following components:
(i) a composition including clarithromycin in admixture with a pharmaceutically acceptable carrier; and
(ii) a composition including rifaximin in admixture with a pharmaceutically acceptable carrier,
wherein the components (i) and (ii) are each provided in a form that is suitable for administration in conjunction with the other.
